(19) United States
(12) Patent Application Publication
(10) Pub. No.: US 2009/0048480 A1
(43) Pub. Date: Feb. 19, 2009

(54) CARDIAC HARNESS DELIVERY DEVICE

(75) Inventors: ALAN R. KLENK, San Jose, CA (US); Joshua Wallin, San Jose, CA (US)

Correspondence Address: FULWIDER PATTON LLP, HOWARD HUGHES CENTER, 6060 CENTER DRIVE, TENTH FLOOR, LOS ANGELES, CA, 90045, US

(73) Assignee: PARACOR MEDICAL, INC., Sunnyvale, CA (US)

(21) Appl. No.: 11/837,619

(22) Filed: Aug. 13, 2007

(51) Int. Cl. A61F 2/00

(52) U.S. Cl. 600/37

(57) ABSTRACT

An apparatus for delivering a cardiac harness onto a heart includes an elongate body and a plurality of elongate push rods longitudinally movable with respect to the elongate body. The elongate body has a tubular housing that is sized to contain the cardiac harness which is removably attached to the push rods. The cardiac harness is releasably attached to the push rods such that advancement of the push rods in a distal direction moves the cardiac hearness from a compacted configuration in the housing to an expanded configuration outside the housing. A deflector is attached to the elongate body and is configured to flare radially outwardly. As the push rods advance out of the housing, the push rods slide over the deflector to more easily and safely advance over the enlarged heart and position the cardiac harness over the heart.

### FIELD OF THE INVENTION

The present invention relates generally to a device for delivering a cardiac harness onto the heart of a patient.

### BACKGROUND OF THE INVENTION

Congestive heart failure (“CHF”) is characterized by the failure of the heart to pump blood at sufficient flow rates to meet the metabolic demand of tissues, especially the demand for oxygen. It has been determined that a passive wrap, or cardiac harness, may increase the efficiency of a heart affected by congestive heart disease. While advances have been made in cardiac harness technology, a satisfactory device for delivering and positioning the cardiac harness onto a patient's heart has yet to be provided.

In one method, access to a patient's heart is achieved through an open chest procedure, wherein the sternum is split and separated to allow access to the heart. The cardiac harness is then positioned over the heart by manual manipulation. Such an open chest procedure is highly traumatic to the patient and, thus, remains a relatively undesirable option for cardiac harness delivery.

Present cardiac harness delivery devices are adapted for use in minimally invasive procedures in which the delivery devices are advanced through a relatively small incision through the body cavity of a patient. Because of the relatively rigid structure and size of such delivery devices, separate introducer devices are used to create an entry path sufficient in size to allow the delivery device to access the heart. In addition, access to the apex of the heart is typically required, in which case an entry path that passes between two ribs is convenient. Importantly, since CHF hearts are enlarged, they have an apex that is rounded which presents a very steep angle of approach when mounting a cardiac harness over the heart.

### SUMMARY OF THE INVENTION

Accordingly, a need exists for a cardiac harness delivery device that overcomes the disadvantages of the prior art in providing access of a cardiac harness delivery device to the heart. The delivery device includes a deflector that contacts the heart and provides an atraumatic guide for the cardiac harness as the harness is mounted onto the heart. The deflector prevents row flipping (either over or under) associated with certain cardiac harness structures and it permits a smooth transition for the harness as it is advanced over the steep angle presented by the enlarged CHF heart.

In one aspect of the invention, an apparatus for delivering a cardiac harness onto a heart includes: an elongate body with a distal portion having a tubular housing sized to contain the cardiac harness in a compacted configuration; a plurality of elongate push rods longitudinally movable with respect to the elongate body; the cardiac harness being removably attached to the elongate push rods; and a deflector for use in deflecting the push rods and the cardiac harness as they are advanced onto the heart in order to provide a smooth transition from the tubular housing where the cardiac harness and push rods are in a compact configuration into an expanded configuration as the cardiac harness and push rods are advanced over the deflector and onto the heart.

In one aspect of the invention, an apparatus for delivering a cardiac harness onto a heart includes: an elongate body with a distal portion having a tubular housing sized to contain the cardiac harness in a compacted configuration; a plurality of elongate push rods longitudinally movable with respect to the elongate body; and a deflector associated with the tubular housing for providing a pathway as the push rods and cardiac harness are advanced out of the housing and onto the heart. In another aspect of the invention, a medical device includes a deflector having a plurality of petals having a distal end and a proximal end, the proximal end of the petals being attached to a ring. The petals taper from a relatively narrow proximal end to a relatively wider distal end. The petals are flexible so that they can be collapsed into a delivery configuration in the housing of a delivery device and flared radially outwardly into a deployed configuration upon advancement out of the delivery device. In one embodiment, the petals are formed from a polymer material such as PEBAX, silicone rubber, polyurethanes, and nylons. At least some of the petals can be loaded with a radiopaque material to enhance visualization of the petals under fluoroscopy, or the polymer material of the petals have a radiopaque material attached thereto in order to enhance visualization under fluoroscopy.

In another aspect of the invention, a medical device includes a deflector having a first ring with a plurality of first petals attached to the first ring, the first petals being spaced apart and forming first gaps between adjacent first petals. The deflector also includes a second ring with a plurality of second petals attached to the second ring, the second petals being spaced apart and forming second gaps between adjacent second petals. Further, the first ring and the second ring are configured to interlock so that the first petals and the second petals overlap when the first ring and the second ring are interlocked. The interlocking rings provide a smooth transition area as the harness is advanced over the deflector.

In another aspect of the invention, an apparatus for delivering a cardiac harness onto the heart includes an elongate body having a proximal portion and a distal portion, with the distal portion having a tubular housing sized to contain the cardiac harness in a compacted configuration. A plurality of elongate push rods are longitudinally movable with respect to the elongate body, and the cardiac harness is releasably connected to the push rods such that advancement of the push rods in a distal direction moves the cardiac harness from the compacted configuration in the housing to an expanded configuration outside the housing. A deflector includes a plurality of flexible petals that are configured to be collapsed into the elongate body in a delivery configuration and flared radially outwardly in a deployed configuration. A deflector sheath in the form of a tubular body fits over the housing and is axially slidable thereon. The deflector sheath retains the deflector until the sheath is withdrawn proximally so that the deflector petals can flare radially outwardly to the deployed configuration. As the push rods and cardiac harness are advanced distally outside the elongate body they slide over the deflector and ease the transition of the push rods and harness expanding as they are advanced over the heart. The cardiac harness is releasably connected to each of the push rods such that advancement of the push rods in a distal direction moves the cardiac harness from the compacted configuration in the housing over the deflector, to an expanded configuration outside the housing so that the harness can be released from the push rods after the harness is pushed onto the heart.

In another aspect, the housing has a substantially circular cross-sectional shape having a diameter. In this aspect, at least a portion of the housing is compressible to a substantially elliptical cross-sectional shape having a minor axis that is less than the diameter. In yet another aspect, the housing has a cross-sectional shape having a first perimeter. A deflector sheath for retaining the deflector has a second perimeter that is greater than the housing first perimeter so that the deflector sheath slidingly extends over at least a distal portion of the housing. At least a portion of the housing and deflector sheath are compressible to a reduced cross-sectional shape having a third perimeter that is less than the first and second perimeter. The deflector also is compressible to conform to the elliptical cross-sectional shape of the deflector sheath to facilitate delivery.

In yet another aspect, the housing has a cross-sectional shape having a first dimension. The first dimension is equivalent to the shortest possible linear distance between any two points on the perimeter of the cross-sectional shape and passing through the center of the cross-sectional shape. In this aspect, at least a portion of the housing is collapsible to a reduced cross-sectional shape having a second dimension that is less than the first dimension. The second dimension is equivalent to the shortest possible linear distance between any two points on the perimeter of the reduced cross-sectional shape and passing through the center of the reduced cross-sectional shape. A deflector sheath and deflector mounted on a distal portion of the housing also are compressible to conform to the housing second dimension reduced cross-sectional shape.

In another aspect, the housing tapers from a first cross-sectional shape at the proximal end of the housing to a second cross sectional shape at the distal end of the housing. In this aspect, the perimeter of the second cross-sectional shape is smaller than the perimeter of the first cross-sectional shape. A deflector sheath slidingly mounted over at least a distal portion of the housing also tapers from the deflector sheath proximal end having the perimeter with the first cross-sectional shape to the distal end having the perimeter with the second cross-sectional shape.

### BRIEF DESCRIPTION OF THE DRAWINGS

These and other features, aspects and advantages of the present invention are described with reference to drawings of a preferred embodiment, which are intended to illustrate, but not to limit, the present invention.

FIG. 1 is a perspective view of a cardiac harness delivery device constructed in accordance with certain features, aspects and advantages of the present invention. The illustrated delivery device comprises a body portion, including an elongate shaft and a housing, and a movable portion, including a control assembly and a plurality of elongate push rods. A cardiac harness is carried by distal end portions of the plurality of push rods.

FIG. 2 is an enlarged, partial cutaway view of a distal portion of the delivery device of FIG. 1 showing the cardiac harness in a compacted configuration within a cavity defined by the housing.

FIG. 3 is a perspective view of the delivery device of FIG. 1 with the movable portion in an advanced position relative to the body portion.

FIG. 4 is an enlarged view of a distal portion of the delivery device of FIG. 1 indicated by line 4-4 of FIG. 3.

FIG. 5 is a perspective view of a deflector including first petals attached to a first ring.

FIG. 6 is a perspective view of a deflector including a plurality of second petals attached to a second ring.

FIG. 7 is a perspective view of a deflector including the first and second petals being interlocked or overlapped with the first ring and second ring interlocked.

FIG. 8 is a partial, enlarged, longitudinal cross-sectional view of the distal portion of the housing depicting the deflector sheath and deflector.

FIG. 9A is an enlarged side elevational view of the delivery device depicting the deflector sheath withdrawn proximally so that the deflector expands into a deployed configuration.

FIG. 9B is an enlarged side elevational view depicting a portion of the delivery device and more specifically depicting the deflector in a delivery configuration compressed within the deflector sheath.

FIG. 10 is an elevational view of a portion of the delivery device depicting the deflector in its deployed configuration on the surface of a heart.

FIG. 11 is an enlarged partial elevational view of the delivery device depicting the deflector in its deployed configuration on the heart.

FIG. 12 is an enlarged partial elevational view of the delivery device depicting the deflector in its deployed configuration on the heart and the push rods, with the cardiac harness attached thereto, being advanced distally out of the housing.

FIG. 13 is an enlarged partial elevational view of the delivery device depicting the push rods advancing distally out of the housing and being deflected radially outwardly by the deflector.

FIG. 14 is an enlarged partial elevational view of the delivery device depicting the push rods further advancing out of the housing and being deflected and guided by the deflector as the push rods advance over the heart.

FIG. 15 is an enlarged partial elevational view of the delivery device depicting the push rods further advancing over the deflector and over the surface of the heart, and thereby advancing the cardiac harness over the heart.

FIG. 16 is an enlarged partial elevational view of the delivery device depicting the push rods further advancing over the deflector and over the heart to advance the cardiac harness over the heart.

FIG. 17 is an enlarged partial elevational view of the delivery device depicting the push rods being withdrawn proximally into the housing over the deflector.

FIG. 18 is an elevational view of the heart depicting the cardiac harness mounted onto the heart and the delivery device being withdrawn.

FIG. 19 is an enlarged partial view of the distal portion of the delivery device depicting the deflector having a flexible cone.

FIG. 20 is an enlarged partial view of the distal portion of the delivery device depicting a deflector having wire form petals bridged by a flexible webbing.

FIG. 21 is an enlarged end view of a distal portion of the delivery device depicting a deflector having wire form petals bridged by a flexible webbing and showing the push rods clocked in relation to the wire form petals.

FIG. 22 is an enlarged partial view of the distal portion of the delivery device depicting a deflector engaging the apex of the heart.

FIG. 23 is an enlarged partial view of the distal portion of the delivery device depicting a deflector having large, overlapping wire form petals bridged by a flexible webbing.

FIG. 24 is an enlarged end view of the distal portion of the delivery device depicting a deflector having large, overlapping wire form petals bridged by a flexible webbing.

FIG. 25 is an enlarged view of the distal portion of the delivery device depicting a deflector having large, overlapping wire form petals bridged by a flexible webbing.

### DETAILED DESCRIPTION OF THE PREFERRED EMBODIMENTS

### Delivery Device

The exemplary figures illustrate a preferred embodiment of a cardiac harness delivery device, which is generally referred to by the reference numeral 30. In a preferred embodiment, the delivery device 30 is configured to releasably support a cardiac reinforcement device (CRD), such as a cardiac harness, and assist in the advancement of the cardiac harness over the heart of a patient. Once the cardiac harness is positioned on the heart, the delivery device 30 preferably is configured to release the harness and be retractable without causing undesired shifting of the cardiac harness relative to the heart.

In the illustrated arrangement, the delivery device 30 permits delivery of a cardiac harness in a minimally invasive manner. That is, preferably the device 30 permits accurate delivery, positioning, and release of the cardiac harness through a relatively small incision in a patient. However, the preferred, or alternative, embodiments of the delivery device 30 may also be used to deliver a cardiac harness in an open chest, or other minimally invasive procedure. Further, an embodiment preferably is configured to enable indirect visualization of at least portions of the device 30 during surgery. For example, portions of the device may be radiopaque so as to be visualized and guided by fluoroscopy or other methods.

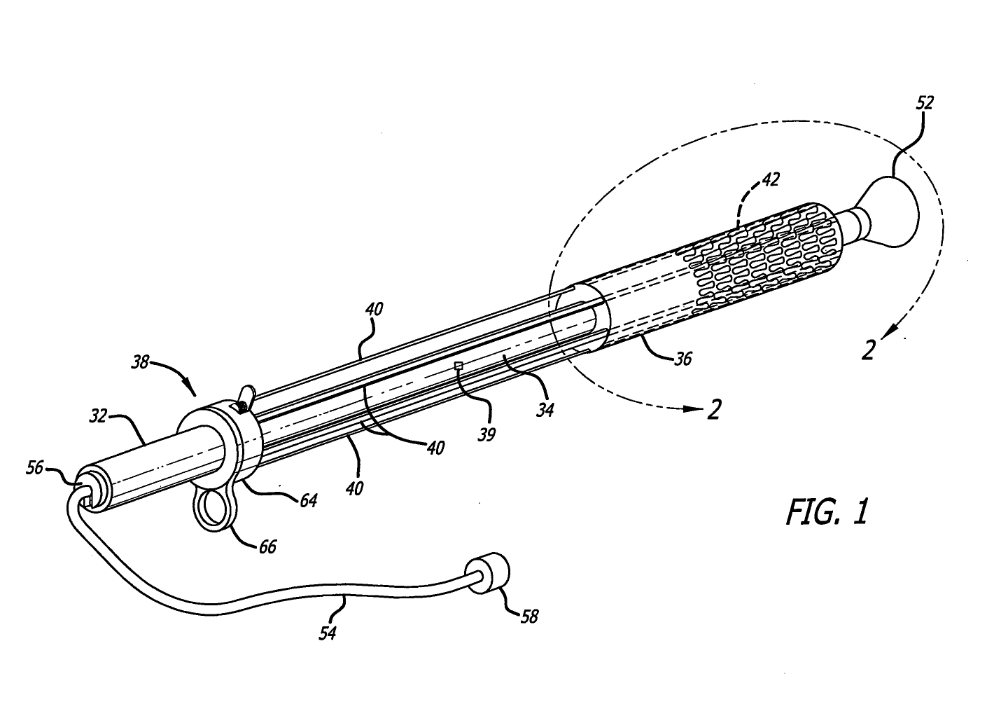

With specific reference to FIG. 1, the illustrated delivery device 30 generally includes a body portion comprised of a handle 32 affixed to the proximal end of a hollow, elongate shaft 34. Preferably, a housing 36 is affixed to a distal end of the elongate shaft 34. The illustrated delivery device 30 also includes a movable portion comprised of a control assembly 38 and a plurality of elongate push rods 40. The control assembly 38 and, thus, the push rods 40, are axially slidable along the shaft 34.

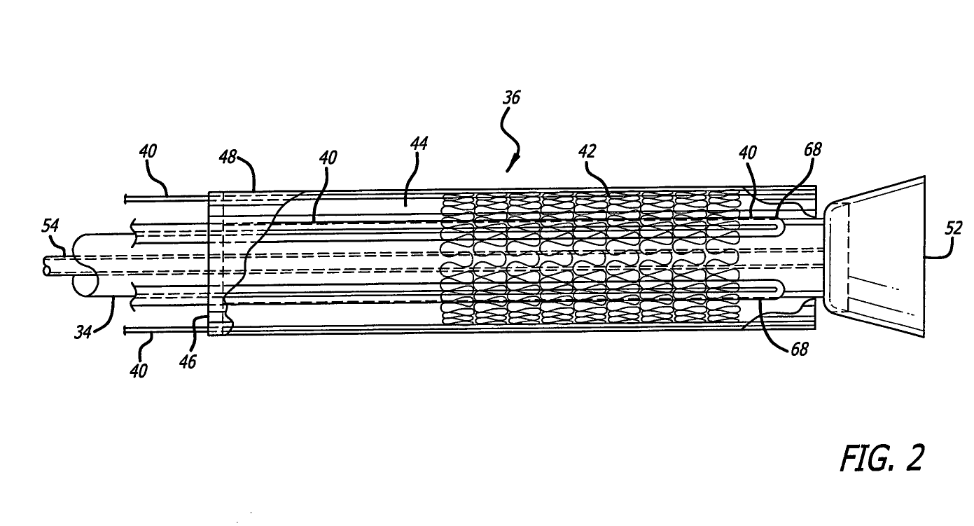

Preferably, the plurality of push rods 40 extend in a distal direction from the control assembly 38 and pass through a housing 36. With reference also to FIG. 2, a cardiac harness 42 is releasably supported on the distal end portions of the elongate push rods 40 in a compacted configuration within the housing 36. Preferably, the cardiac harness 42 comprises an elastic sleeve configured to fit around the heart and to exert a compressive force on the heart. In the illustrated embodiment, the harness 42 comprises several interconnected rows of undulating elastic members. Preferred cardiac harnesses are described in greater detail U.S. Pat. No. 6,702,732; U.S. Pat. No. 6,723,041; U.S. Pat. No. 7,189,203; U.S. application Ser. No. 10/287,723, filed Oct. 31, 2002; and U.S. application Ser. No. 10/656,722, filed Sep. 5, 2003, the entirety of each of which are incorporated by reference herein. It is to be understood that aspects of the delivery device 30 discussed herein can be used in connection with several other types of cardiac harnesses.

The term “cardiac harness” as used herein is a broad term that refers to a device fit onto a patients heart to apply a compressive force on the heart during at least a portion of the cardiac cycle.

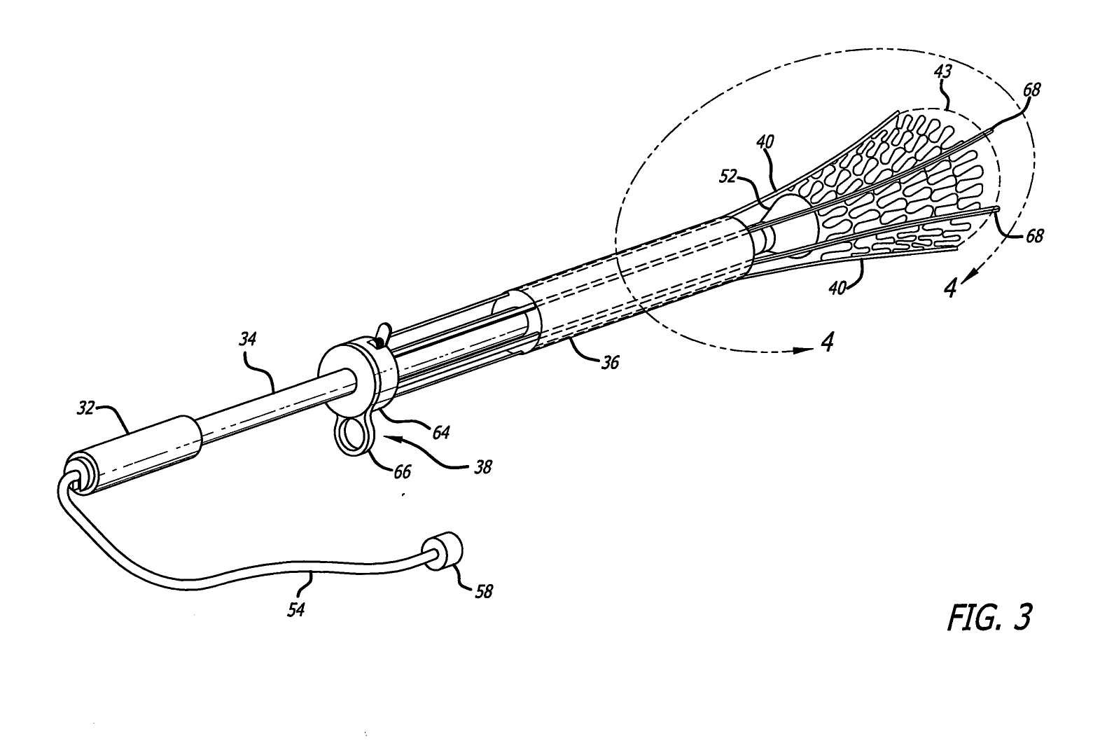

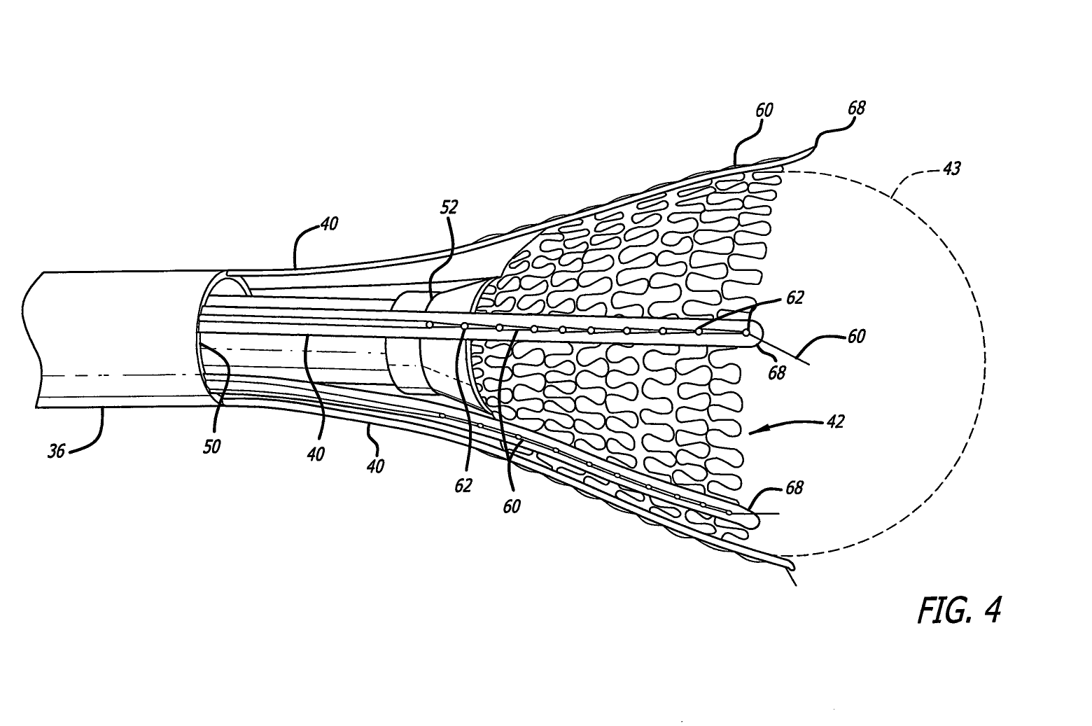

The control assembly 38 and plurality of push rods 40 are movable axially with respect to the shaft 34 from the retracted position, as illustrated in FIGS. 1 and 2, to an advanced, or deployed position, as illustrated in FIGS. 3 and 4. Thus, the delivery device 30 is configured to deploy the cardiac harness 42 from a compacted configuration within the housing 36 to an expanded configuration outside of the housing 36 thereby delivering the cardiac harness 42 onto a heart 43 (illustrated schematically in FIGS. 3 and 4), as is described in greater detail below. Delivery of the cardiac harness to the heart and mounting it onto the heart is described in more detail in U.S. Pat. No. 7,189,203, which is incorporated by reference herein.

The handle 32 is fixed to the shaft 34 in the illustrated embodiment. However, it is to be understood that in other arrangements the handle 32 may be movable relative to the shaft 34 along with the control assembly 38. Additionally, another embodiment may not employ a handle 32. Further, with reference to FIG. 1, a stop 39 preferably is provided on the shaft 34. The stop 39 comprises a raised portion that engages the control assembly 38 so that the assembly 38 cannot move distally over the shaft 34 beyond the stop 39. As such, the harness 42 is not advanced too far over the heart 43.

With reference again to FIG. 2, the housing 36 preferably is a relatively thin-walled, tubular member. Desirably, the housing 36 is supported substantially concentric with the shaft 34 to define an interior cavity 44 between an inner surface of the housing 36 and an outer surface of the shaft 34. Preferably, the cavity 44 is sized and shaped to contain the cardiac harness 42 in a compacted configuration therein.

As indicated above, preferably the device 30 is configured to deliver the cardiac harness 42 in a minimally invasive procedure. Accordingly, a preferred housing 36 has a nominal outer diameter of less than about 5.1 cm (2 inches), more preferably, less than about 3.2 cm (1.25 inches). Preferably, the housing 36 is flexible such that its transverse cross-sectional shape may be collapsed or compressed as needed to advance through a minimally invasive surgical entry path, as described in greater detail below. In the illustrated embodiments, the housing 36 is generally cylindrical in its relaxed or uncompressed condition. It is to be understood that, in another preferred embodiment, the housing is substantially elliptical in its relaxed condition such that the housing may have a cross-section with major axis and minor axis. This configuration may be especially beneficial for advancing the housing through body passages having relatively narrow clearance, such as advancing the housing between the ribs.

With continued reference to FIG. 2, a base portion 46 of the housing 36 preferably defines a closed end of the cavity 44 and supports the housing 36 relative to the shaft 34. The base end 46 may be secured to the shaft 34 by mechanical fasteners, adhesives or other suitable methods apparent to one of skill in the art. In one embodiment, the base end 46 is rotatable relative to the shaft 34. Preferably, the distal end of the housing is open to define an open, distal end of the cavity 44 to permit the cardiac harness 42 to be advanced from the cavity 44.

Preferably, an inner surface of the housing 36 defines a plurality of channels 50 (FIG. 4) extending axially throughout the length of the housing 36. Each of the channels 50 preferably is sized and shaped to slidably receive one of the plurality of push rods 40. Thus, preferably, the number of channels 50 is equal to the number of push rods 40. Further, each channel 50 preferably opens into a cavity 44 along at least a portion of the length of the channel 50.

In the embodiments illustrated, eight push rods 40 and eight channels 50 are provided and are substantially equally spaced around the circumference of the housing 36. A greater or lesser number of push rods 40 and channels 50 may be provided as appropriate to support and deploy a cardiac harness. In an additional arrangement, the channels 50 may be omitted and the push rods 40 may simply be restrained from moving radially outwardly by an outer wall 48 of the housing 36. Other suitable arrangements to guide the push rods 40 and house the cardiac harness 42 may also be used.

With continued reference to FIGS. 1-4, the delivery device 30 preferably includes a positioning arrangement configured to hold the delivery device 30 in a desired position relative to the heart 43. In the illustrated arrangement, the positioning arrangement comprises a suction cup member 52 supported on a distal end of the shaft 34. A tube 54 extends through the shaft 34 and is connected to the suction cup member 52. A distal end of the tube 54 opens into an interior space defined by the suction cup member 52. The proximal end of the tube 54 includes a connector 58 that allows connection of the tube 54 to a pump member such as a syringe or other source of vacuum. Accordingly, once the delivery device is properly positioned, air may be withdrawn from within the tube 54 to create a vacuum condition within the interior space of the suction cup member 52, thereby permitting the suction cup member 52 to securely hold the heart of a patient.

In one embodiment, the tube 54 and suction cup member 52 are not rigidly affixed to the shaft 34 so that the shaft 34 may be moved relative to the tube 54 and suction cup 52. In another embodiment, the shaft 34 and a proximal end of the suction cup 52 are threaded so that the suction cup may be threaded onto the shaft. In still other embodiments, other structure may be used to releasably connect the suction cup to the shaft.

Preferably, the cardiac harness 42 is secured to a distal portion of each of the plurality of push rods 40 by a flexible line that is configured into a releasable stitch, such as described in U.S. Pat. No. 7,189,203, the entirety of which is incorporated by reference herein. Desirably, as shown in FIG. 4, the flexible line 60 passes through a plurality of openings 62 in the distal portion of the push rod 40 and is arranged into a series of interconnected loops that are releasable by actuation of the control assembly 38 in a manner described in greater detail below. Release of the interconnected loops, in turn, releases the cardiac harness 42 from the push rods 40.

With particular reference to FIGS. 1 and 3, the control assembly 38 preferably includes a substantially cylindrical body portion 64 and a release member 66. A portion of the release member 66 preferably is received within a cavity of the body portion 64. An exposed pull portion of the release member 66 extending outwardly from the body portion 64 is generally annular in shape, such that a user of the delivery device 30 can grasp the release member 66 with one or more fingers extending through the a hole defined by the annular shape. As the release member 66 is pulled away from the body portion 64 of the control assembly 38, the release member 66 pulls on the flexible lines 60 such that the interconnected loops of the releasable stitch are unraveled.

### Deflector

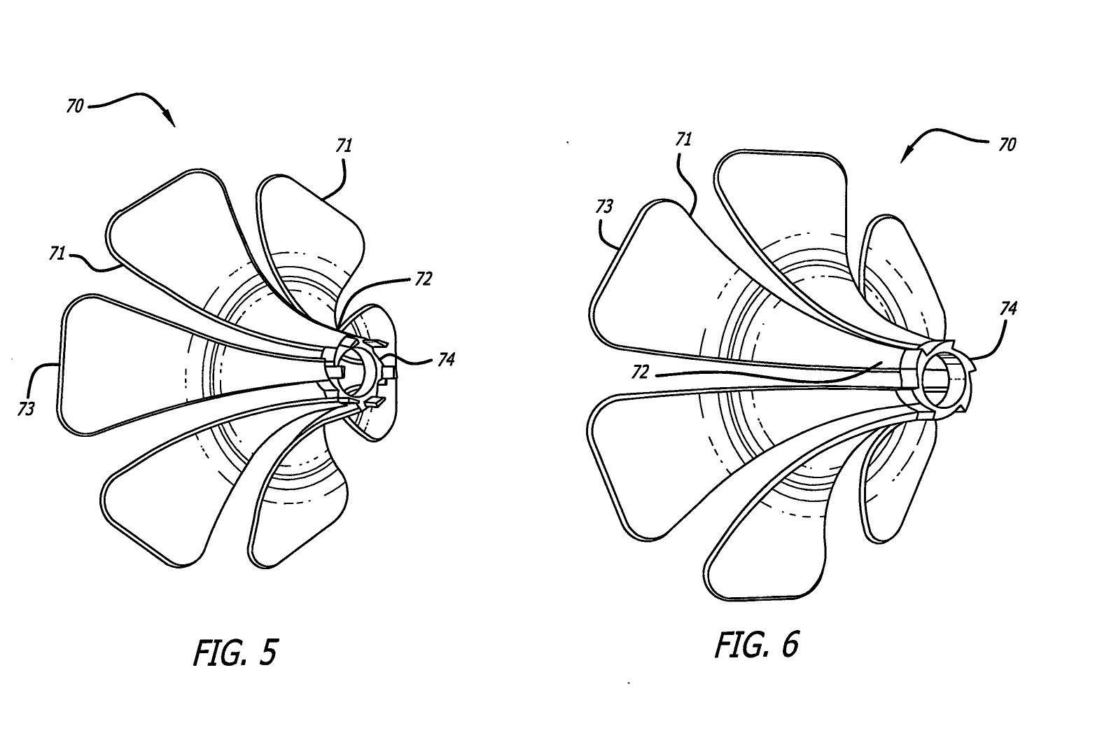

In one aspect of the invention, as shown in FIGS. 5-7, a medical device includes a medical apparatus for delivering a cardiac harness onto the heart. The medical device includes a deflector 70 having a number of petals 71 with a proximal end 72 and a distal end 73 and a ring 74, whereby the proximal end of the petals are attached to the ring. Typically the petals 71 will taper from a relatively narrower proximal end to a relatively wider distal end. The petals are flexible so that they can be collapsed into a delivery configuration and are adapted flare radially outwardly into a deployed configuration. In one embodiment, the cardiac harness is compressed into a tubular housing and releasably attached to push rods that are longitudinally movable relative to the housing. The cardiac harness is advanced out of the housing by the push rods, which engage the deflector so that the distal ends of the push rods are deflected radially outwardly in order to more easily conform to the surface of the heart and to protect the heart from any trauma associated with the advancing push rods.

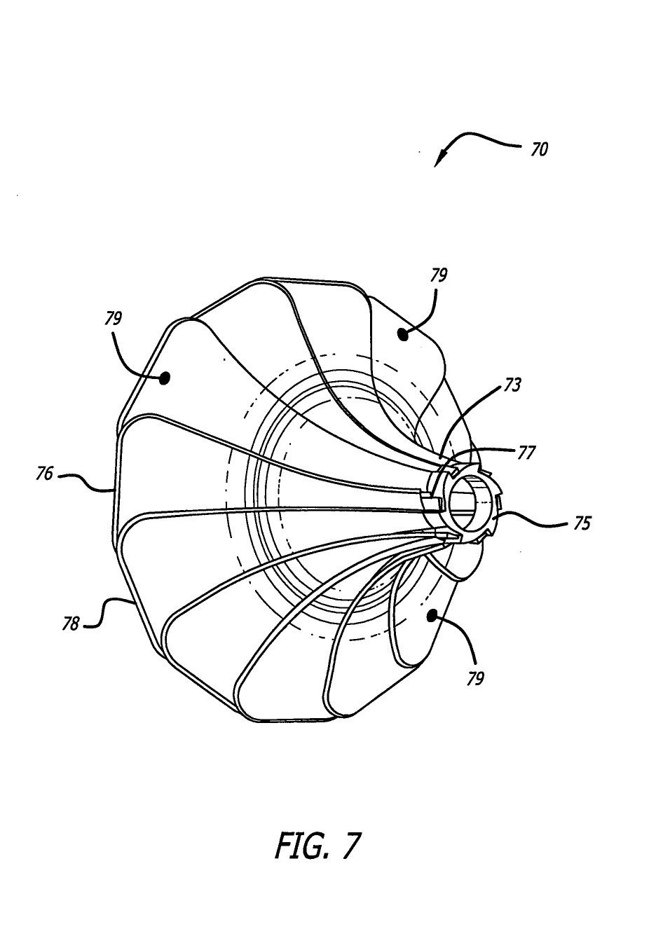

In another embodiment, as shown in FIGS. 5-7, a medical device includes a deflector 70 having a first ring 75 having first petals 76 attached to the first ring, and a second ring 77 having second petals 78 attached to the second ring 77. The first ring 75 and the second 77 ring are adapted to interlock so that the first petals 76 and the second petals 78 overlap as shown in FIG. 7. Since the first petals 76 and the second petals 78 overlap, and due to the flexibility of the petals, the petals can slidingly engage each other so that they can be compressed into a delivery configuration and will flare radially outwardly into a deployed configuration, as described below. In one embodiment, the deflector 70 is firmly attached to the delivery device 30 by inserting the proximal end of the suction cup 52 through the assembled rings 75,77 and threading the proximal end of the suction cup 52 onto corresponding threads in shaft 34. Thus, deflector 70 and suction cup 52 are firmly attached to the shaft 34.

In one embodiment, the deflector 70 includes a plurality of petals 71 that number in the range of from four to twenty petals. The petals can be formed of a polymer material, polyamides, polyamide copolymers such as PEBAX (a polyether block amide), silicone rubber, polyurethanes, and nylons. The petals and ring can be injection molded by known techniques. The petals also can be formed from a metallic material, such as nitinol or a combination of a nitinol and polymer webbing. At least some of the petals are loaded with a radiopaque material to enhance visualization of the device under fluoroscopy, or have a radiopaque material embedded in the petal. For example, radiopaque plugs, beads or wires 79 made from high density metals can be embedded in the petals 71 to enhance the visability of the petals under fluoroscopy or by other imaging means.

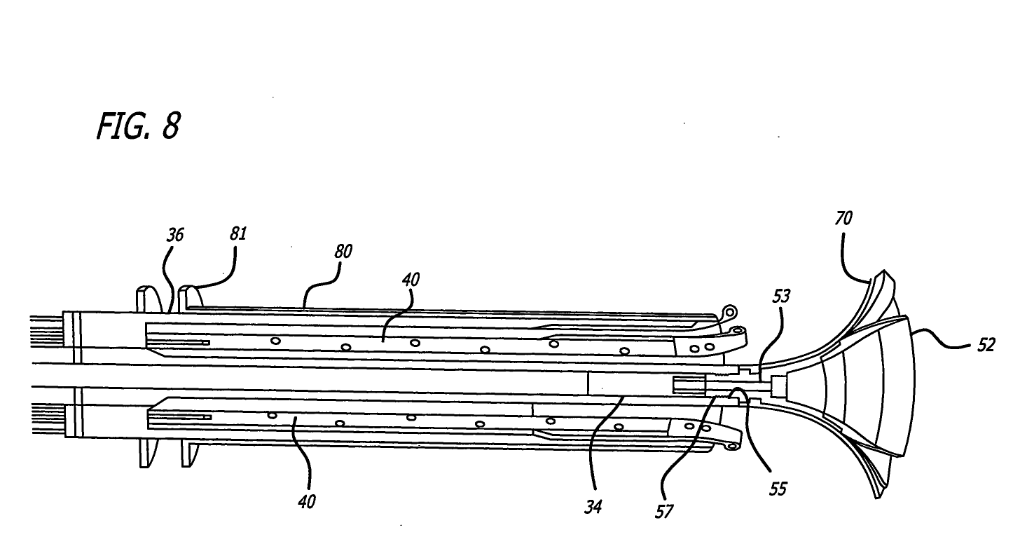

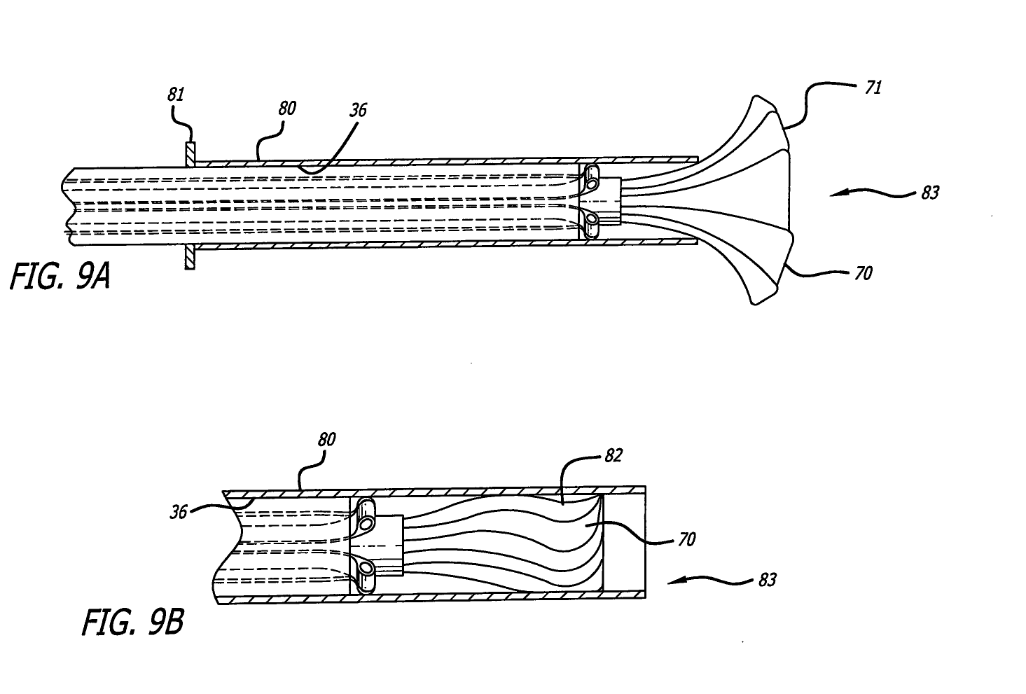

In further keeping with the invention, as shown in FIGS. 8 and 9A and 9B, deflector 70 is housed in a deflector sheath 80, which is preferably a clear plastic tube having a flange 81 at its distal end. Preferably, deflector 70 is attached to the delivery assembly so that it remains axially stationary during use. In this embodiment, the suction cup 52 has a shaft 53 that extends through the rings 75,77 of the deflector 70. Screw threads 55 on shaft 53 matingly engage screw threads 57 on housing shaft 34 so that the suction cup 52 is screwed onto housing shaft 34 and in the process attaches the deflector 70 over the suction cup 52. The deflector sheath 80 is sized so that it fits over a distal portion of the delivery device 30 and can slide axially relative to the delivery device 30 and the deflector 70. In this manner, the doctor can push on the flange 81 in a distal direction so that the deflector sheath 80 can be pushed over the deflector 70 thereby compressing the petals 71,76,78 into a delivery configuration 82, as shown in FIG. 9. As the deflector sheath 80 is pushed distally over the deflector, the petals 71,76,78 slidingly overlap to a smaller diameter until they reach the delivery configuration 82. By pulling back on flange 81 in a proximal direction, the deflector sheath 80 slides axially over the delivery device 30 so that the deflector 70 emerges from the deflector sheath and the petals 71,76,78 slidingly open over each other to flare radially outwardly into a deployed configuration 83. The petals are biased radially outwardly so that they automatically expand radially outwardly as the deflector sheath 80 is withdrawn axially in a proximal direction.

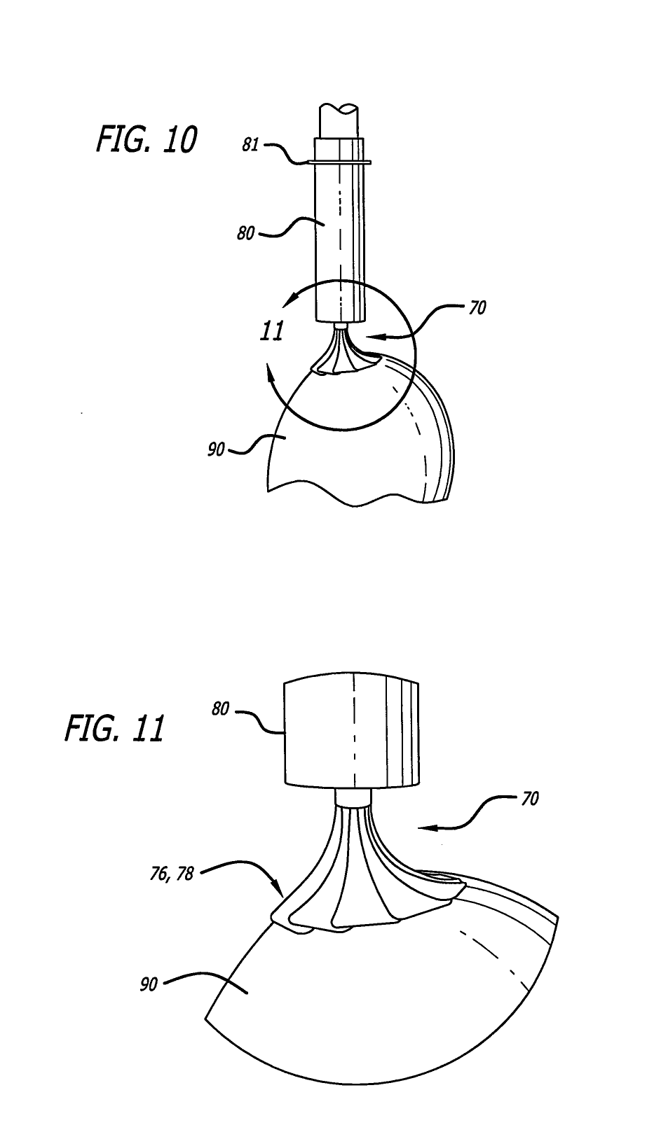

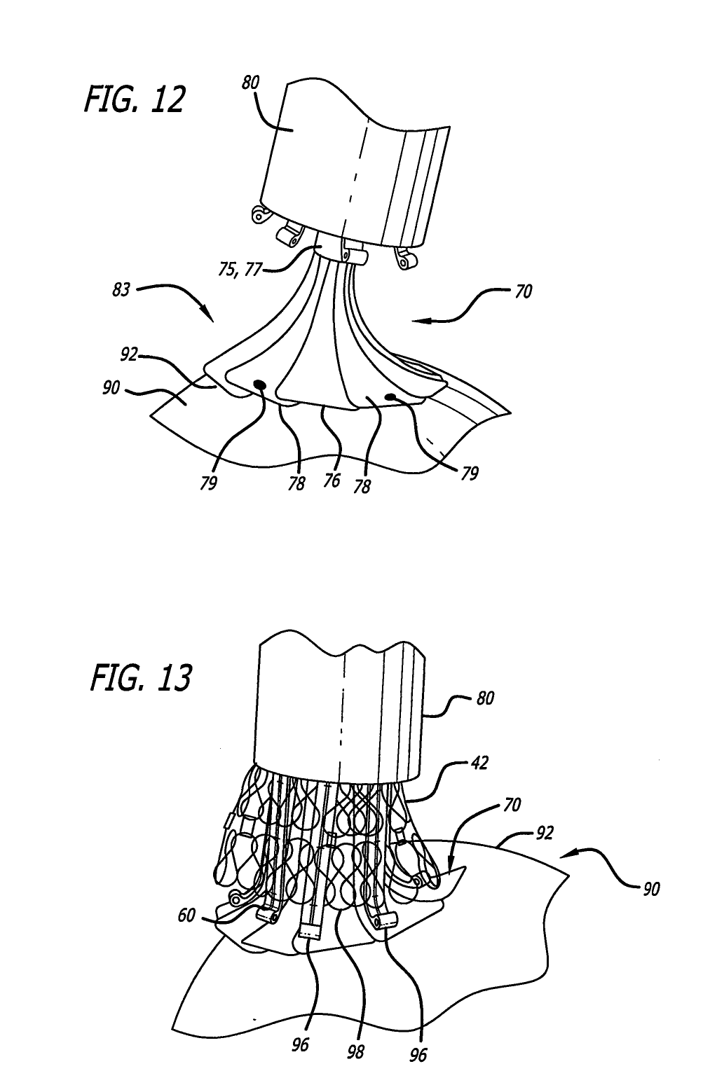

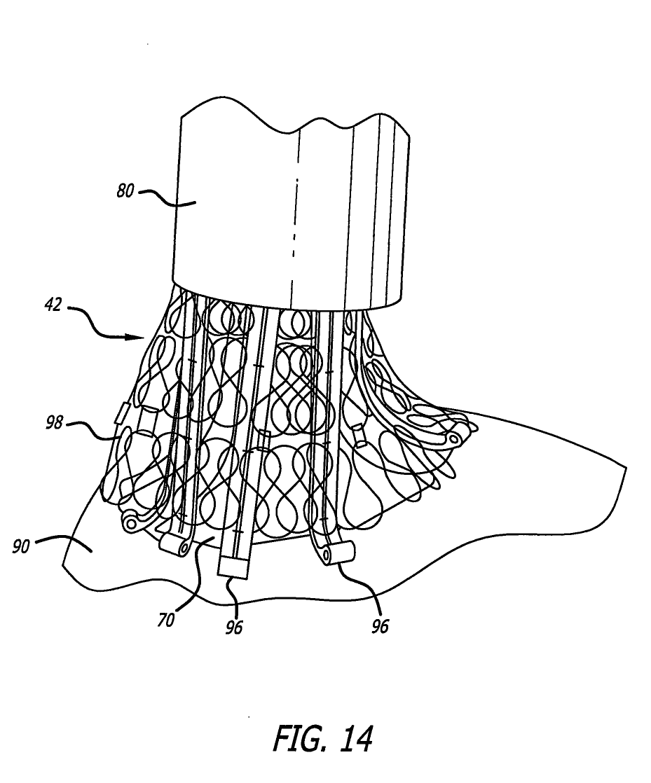

FIGS. 10-18 illustrate the use of a delivery device 30, preferably configured substantially as described above, to deliver a cardiac harness 42 onto a heart 90. Preferably, the delivery device 30 is configured to locate and grasp the heart 90, accurately position the cardiac harness 42 onto the heart 90, and permit withdrawal of the delivery device 30 without disturbing the positioning of the cardiac harness 42. As shown more specifically in FIGS. 10 and 11, the deflector sheath 80 has been withdrawn proximally thereby exposing the deflector 70, which has flared radially outwardly and is in contact with the heart 90 (in this case an schematic representation of the heart). Not visible in FIGS. 10 and 11 is suction cup 52 which is used to securely fasten, via a vacuum, the delivery device 30 to the apex portion 92 of the heart 90. The deflector 70 extends over the suction cup 52.

With reference to FIG. 4, preferably, the suction cup 52 of the delivery device 30 engages an apex portion 92 of the heart 90, which is illustrated schematically in FIG. 4. The distal end of the delivery device 30 may access the heart 90 through any suitable method, but preferably through a minimally invasive procedure. In FIGS. 4 and 10-18, the pericardial sac or pericardium surrounding the heart is omitted for ease of illustration.

A pump device, such as a syringe, is connected to the tube 54 through the connector 58. Desirably, the syringe is connected to the tube 54 with the plunger in a compressed position. Once connected, the plunger is retracted to create a vacuum condition within the tube 54 and, thus, within the space defined by the interior of the suction cup member 52. Due to the vacuum condition, the suction cup member 52 grasps the apex 92 such that the heart 90 is held in a desired position relative to the delivery device 30.

With reference next to FIGS. 12-16, once the delivery device 30 has been properly secured to the apex portion 92 of the heart 90, the control assembly may be advanced, relative to the shaft, toward the heart 90. The plurality of push rods 40 are advanced toward the heart 90 with the control assembly thereby advancing the cardiac harness 42 from its compacted configuration within the housing onto the heart 90 in a direction from the apex portion 92 to the base portion 94. As shown, the harness 42 preferably stretches elastically to fit over the heart. However, it is to be understood that a substantially non-elastic harness embodiment can also be delivered by this device.

The plurality of push rods 40 splay outwardly to conform to the shape of the heart 90 as they are advanced over the deflector 70. Preferably the tips 96 of the push rods 40 are canted at an outward angle relative to the remainder of the push rod 40 such that contact of the tip 96 with the deflector 70 allows a smooth and atraumatic transition of the push rods 40 onto the heart 90. As shown in FIG. 12, even if the delivery device 30 is off center relative to the apex 92, the deflector 70 will re-direct the push rods 40 to protect the heart tissue and advance the harness onto the heart.

An important feature of the deflector is that as the cardiac harness is advanced from the housing onto the heart, the deflector 70 prevents the first rows 98 of the cardiac harness 42 from catching on the heart and either flipping under or flipping over thereby causing an undesirable configuration for advancing the harness. Further, the deflector 70 also prevents the push rods 40 from prolapsing or collapsing due to the severe delivery angle as the push rods are advanced distally over the apex 92 of the heart.

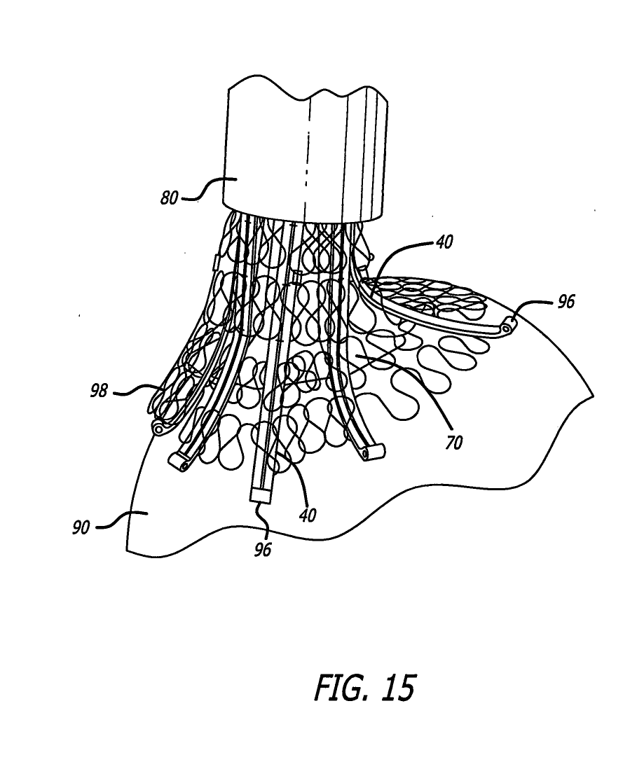

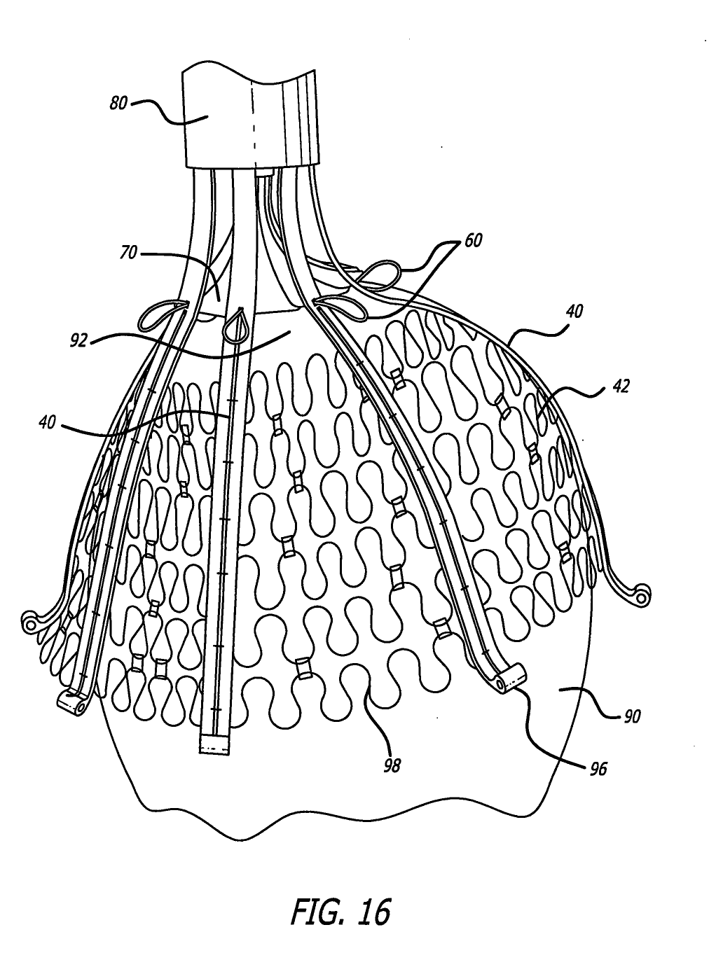

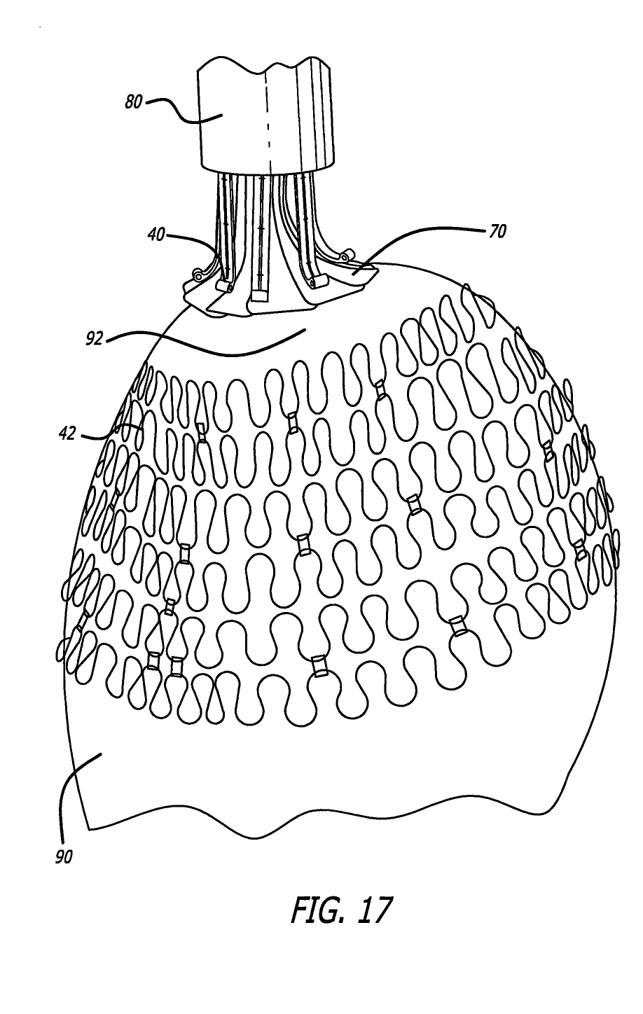

With reference to FIGS. 15 and 16, the control assembly continues to be advanced until the cardiac harness 42 is properly positioned on the heart 90. Once the cardiac harness 42 is properly positioned, the release member is pulled away from the body portion of the control assembly so that the cardiac harness 42 is released from the push rods 40.

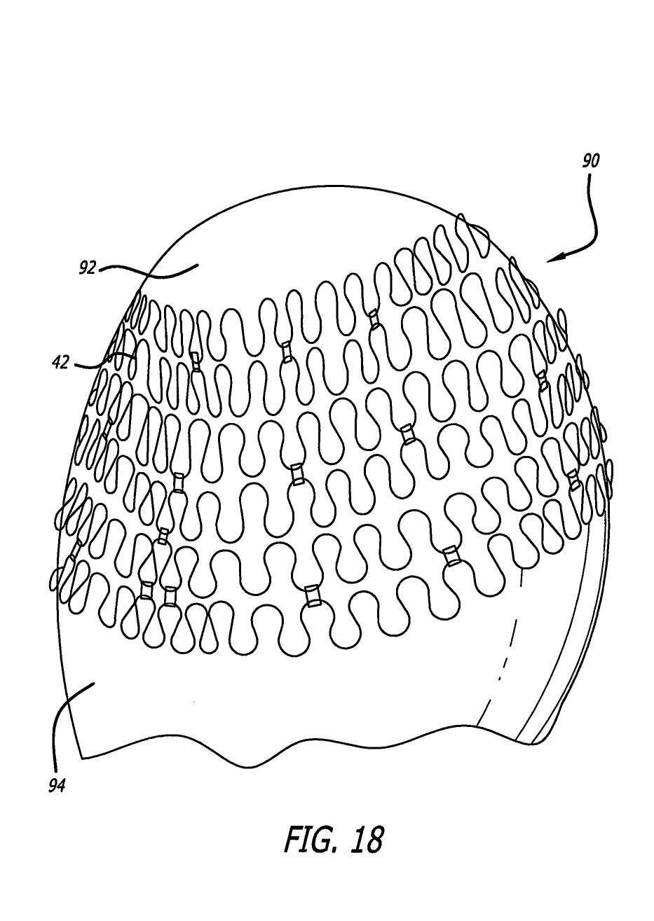

With reference to FIGS. 17 and 18, once the cardiac harness 42 has been released from the plurality of push rods 40, the generally-elastic harness preferably contracts onto the heart. The control assembly is then retracted relative to the shaft to retract the plurality of push rods 40 from the cardiac harness 42, which remains on the heart 90. As noted above, preferably, the push rods 40 are configured such that retraction of the push rods 40 does not tend to pull the cardiac harness 42 from its desired position on the heart 90. Specifically, in the illustrated embodiment, the outwardly canted tips 96 of the push rods 40 help prevent the push rods 40 from exerting a pulling force on the cardiac harness 42. Once the plurality of push rods have been fully retracted from the cardiac harness 42 and the heart 90, the one-way valve within the connector may be opened to release the vacuum condition within the suction cup member 52. As a result, the delivery device 30 may be removed from the heart 90, as the suction cup member 52 is no longer grasping the heart 90. Thus, the delivery device 30 is retracted from the heart, leaving the cardiac harness 42 in place.

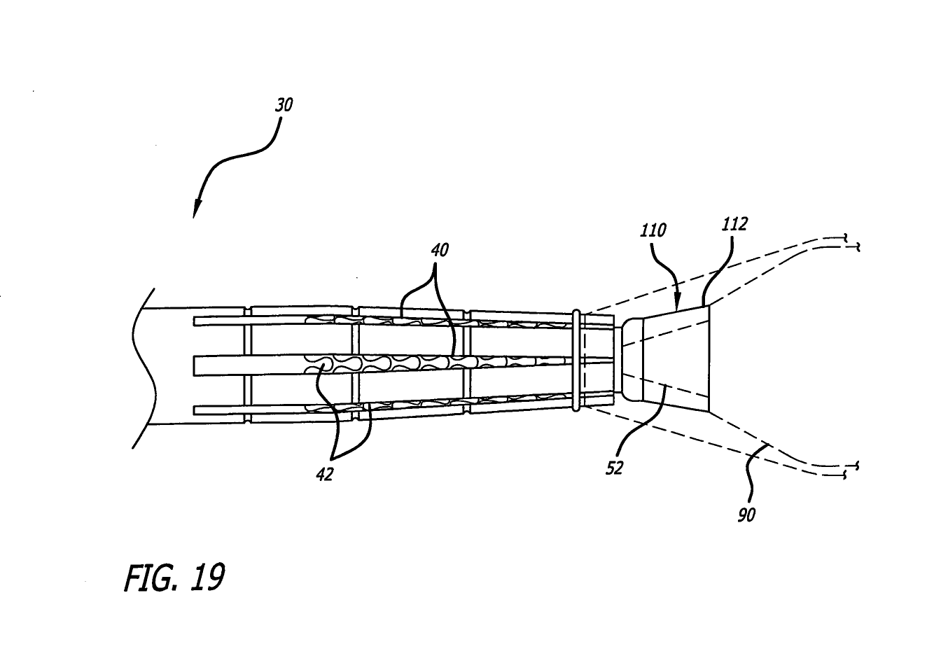

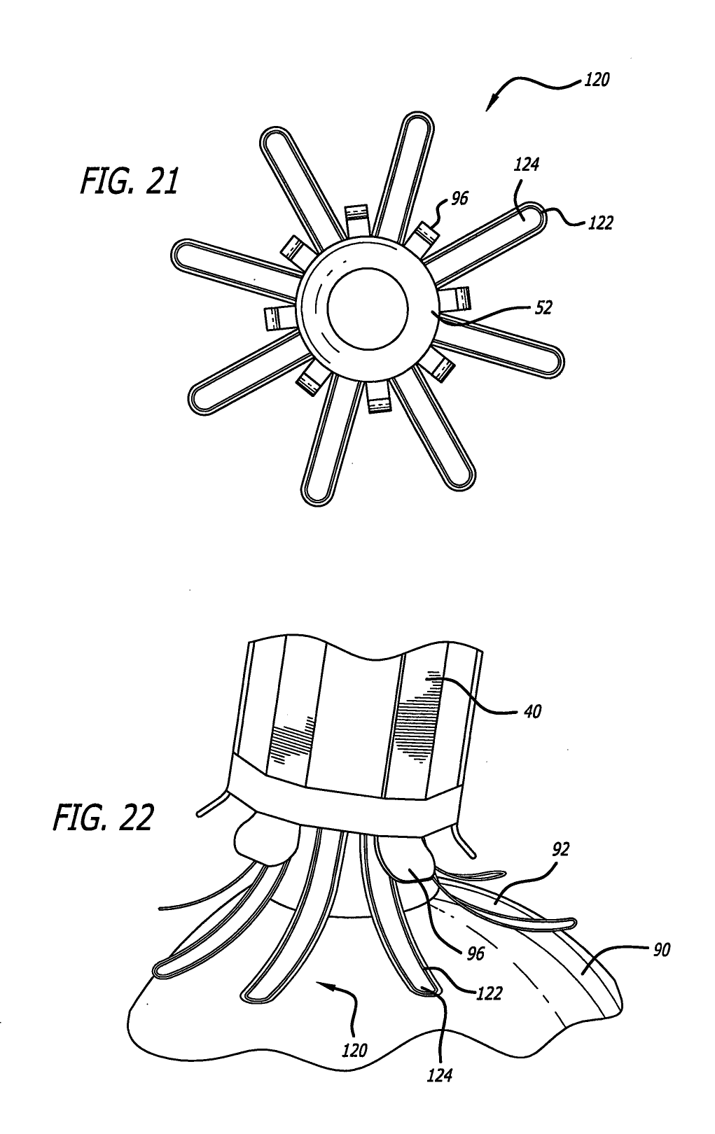

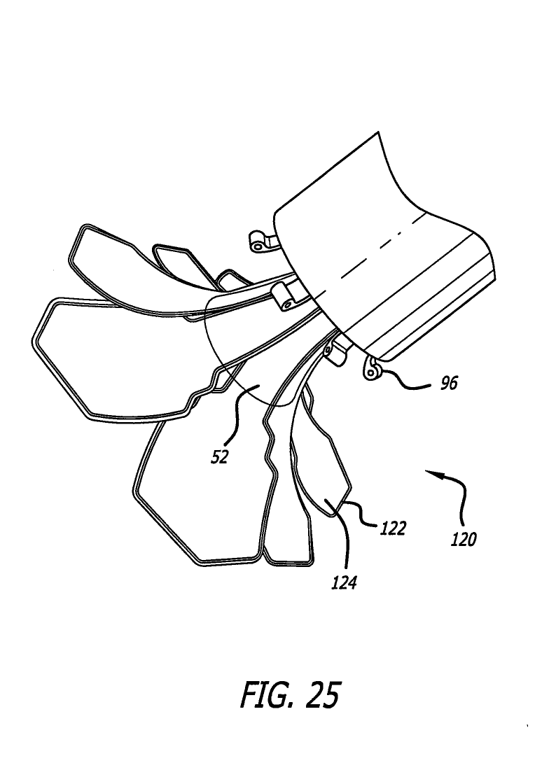

An alternative embodiment of the deflector is shown in FIGS. 19 and 20-25. With reference to FIG. 19, deflector 110 is substantially similar to the deflector previously described with reference to FIGS. 5-18. Deflector 110 includes a flexible cone 112 that flares radially outwardly, but does not have petals like the previously described deflector. In this embodiment, the flexible cone 112 fits over the suction cup 52 and is a substantially solid material formed from any of the polymers previously described such as PEBAX, silicone rubber, polyurethane, or nylons. As previously described, a deflector sheath 80 (not shown) extends over deflector 110 to compress it into a delivery configuration. Because the flexible cone 112 is somewhat compliant, it will collapse and wrinkle up to compress into a delivery configuration. When the deflector sheath 80 is withdrawn proximally, deflector 110 will expand radially outwardly and return to its flexible cone 112 configuration in order to assist in the delivery of the cardiac harness as previously described.

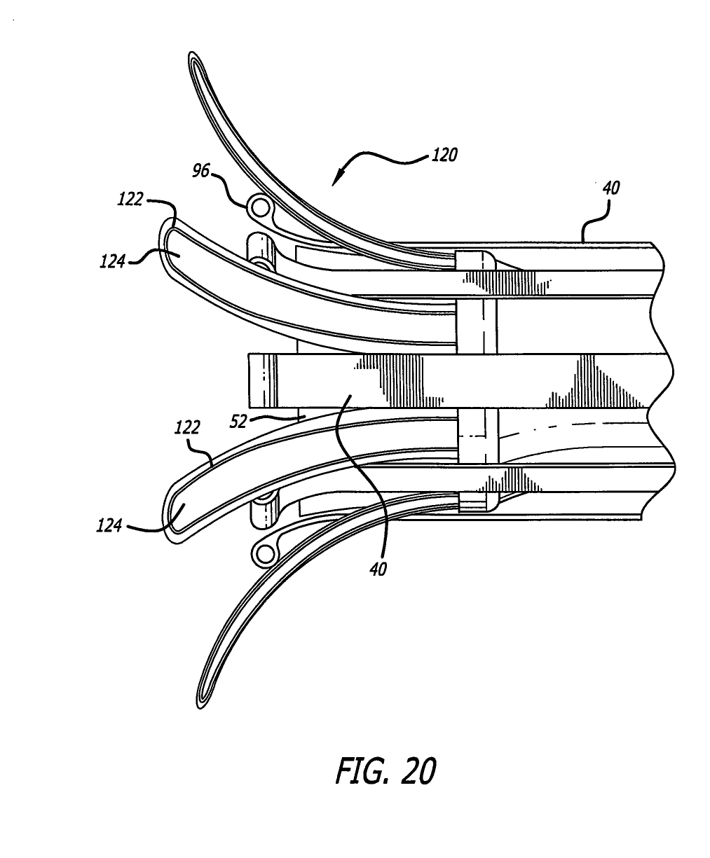

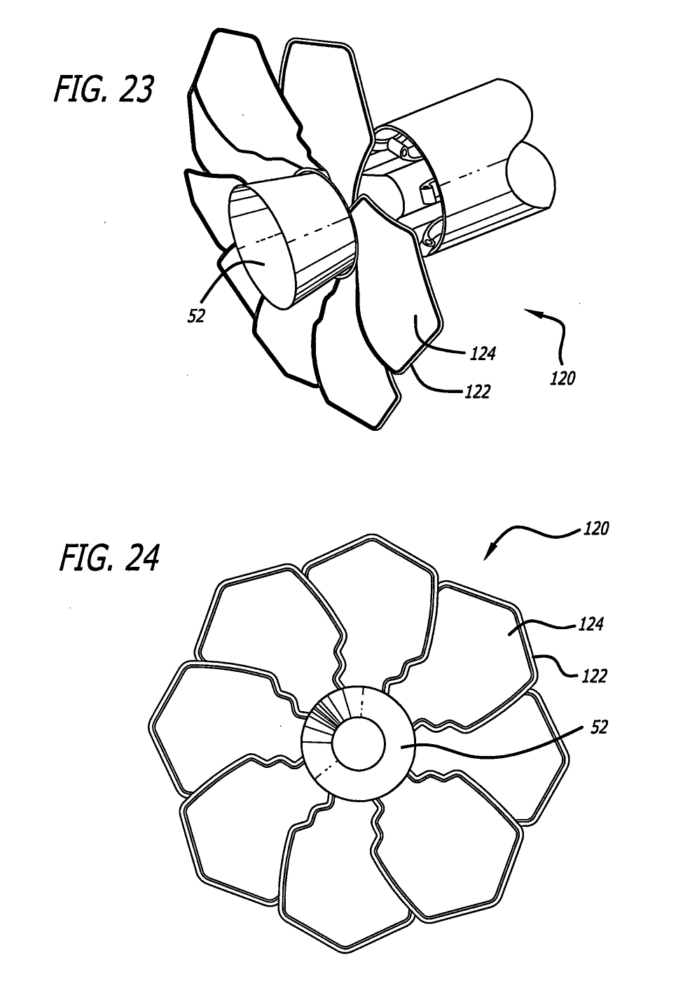

In another embodiment of the deflector, as shown in FIGS. 20-25, a deflector 120 is comprised of a number of wire form petals 122 shaped in a hairpin shape or similar shape and flared radially outwardly. The wire petals have a flexible webbing 124 that includes any type of polymer material previously described. In this embodiment, a 0.015 inch diameter, nitinol, shape set wire can be used to form wire petals 122 to support webbing 124. The webbing 124 can be coated or molded onto the wire petals 122 in any known manner. Deflector 120 operates similar to that discussed above for the embodiments in FIGS. 5-18. In this embodiment, the push rods 40 are clocked to correspond to the spaces between wire form petals 122. As the cardiac harness is advanced out of the delivery device as previously described, the tips 96 of the push rods will extend into the spaces between the wire form petals 122 so that the cardiac harness will engage and slide along wire form petals 122 and webbing 124 thereby deflecting radially outwardly to provide a smooth transition onto the surface of the heart. As shown in FIGS. 23-25, the wire form petals 122 overlap, similar to that shown in FIGS. 5-18.

As discussed above, the housing 36 may have a collapsible cross-sectional shape. To facilitate insertion of the delivery device 30 through a minimally invasive surgical entry path, the distal end of the housing may be compressed or collapsed. To facilitate advancement through a narrow passage in a minimally invasive surgical entry path, such as between two ribs of a patient, the housing may be flattened to an oval or substantially elliptical cross-section with a minor axis and major axis. Likewise, the deflector sheath 80 and the deflector 70 also can be formed of a compressible material and be collapsed along with the distal end of the housing to facilitate advancement through a narrow passage in a minimally invasive surgical entry path, such as between two ribs of a patient. As the housing 36 and deflector sheath 80 and deflector 70 are advanced past a narrow passage, they can return to a circular cross-sectional shape and portions of the housing adjacent to the narrow passage flatten to allow further advancement of the housing. It will be appreciated that, compared to a rigid housing, a housing with a collapsible cross-section shape places less stress on tissues and bones along the minimally invasive surgical path and, thus, is likely to result in lower incidence of injury or trauma.

It is to be understood that other cross-sectional shapes may be achieved by compressing a collapsible housing. With any cross-sectional shape, when it is desired to advance the housing between two ribs or other narrow passage of a minimally invasive surgical entry path, the minimum cross-sectional dimension is preferably less than a distance across the narrow passage.

While the illustrated embodiments shown in FIGS. 10-25 have a housing with flexible push rods separated by gaps, it will be appreciated by persons of skill in the art that other housing structures may be used resulting in a collapsible cross-sectional shape. For example, it is contemplated that a housing may comprise a thin-walled sleeve configured to fold or stretch along a length of the sleeve.

Although the delivery device 30 is especially well suited for use in a minimally invasive delivery procedure, the device 30 may also be used for open chest procedures, wherein the sternum of the patient is split to provide access to the heart 90. In addition, although the device 30 described herein utilizes a plurality of push rods 40, other suitable structures may also be used to support the cardiac harness 42 when being advanced over the heart. For example, an expandable sleeve can serve as a support structure. Furthermore, it is to be understood that a cardiac harness 42 may be releasably supported in an expanded, or substantially expanded, configuration to a variety of support structures by the releasable stitch referred to herein, or by a similar releasable stitch arrangement.

In the embodiments disclosed herein, the illustrated cardiac harness 42 is formed of several rows of elastic elements. The illustrated harness comprises undulating wire arranged in several adjacent rings, each of which comprises an elastic row. As illustrated, the harness 42 is releasably attached to the push rods by a stitch being wound around some or all of the rows. Of course, it is to be understood that aspects of the present invention can be employed with harnesses having different structure than the illustrated harness, which is included for example only. For example, any harness having one or more openings that could accommodate the releasable stitch could be used such as, for example, a harness formed of a woven or non-woven fibrous material and/or a harness formed of a mesh, honeycomb or other type of material.

Although the present invention has been described in the context of a preferred embodiment, it is not intended to limit the invention to the embodiment described. Accordingly, modifications may be made to the disclosed embodiment without departing from the spirit and scope of the invention. It is also contemplated that various combinations or subcombinations of the specific features and aspects of the embodiments can be combined with or substituted for one another in order to form varying modes of the invention. Accordingly, the invention is intended to be defined only by the claims that follow.

What is claimed:

1. A medical device, comprising:
a deflector having a plurality of petals having a distal end and a proximal end;
a ring, the proximal end of the petals being attached to the ring;
the petals tapering from a relatively narrower proximal end to a relatively wider distal end; and
the petals being flexible so that the petals can be collapsed into a delivery configuration and flared radially outwardly into a deployed configuration.

2. The medical device of claim 1, wherein the plurality of petals number in the range from four to twenty petals.

3. The medical device of claim 1, wherein the petals are formed from a polymer material.

4. The medical device of claim 3, wherein the polymer material is taken from the group of polymers including polyamides, polyamide copolymers such as PEBAX, silicone rubber, polyurethanes, and nylons.

5. The medical device of claim 1, wherein at least a portion of the petals are formed from a metallic material.

6. The medical device of claim 1, wherein at least some of the petals are loaded with a radiopaque material to enhance visualization of the device under fluoroscopy.

7. The medical device of claim 6, wherein the radiopaque material is barium sulfate.

8. The medical device of claim 1, wherein a radiopaque material is attached to at least some of the petals to enhance visualization of the device under fluoroscopy.

9. The medical device of claim 1, wherein the distal ends of the petals are biased radially outwardly to form a flared configuration.

10. The medical device of claim 1, wherein at least a portion of each petal overlaps with adjacent petals.

11. The medical device of claim 1, wherein adjacent petals slide over each other when the deflector is compressed into the delivery configuration and expanded to the deployed configuration.

12. A medical device, comprising:
a deflector having a first ring with a plurality of first petals attached to the first ring, the plurality of first petals being spaced apart forming first gaps between adjacent first petals;
the deflector also having a second ring with a plurality of second petals attached to the second ring, the plurality of second petals being spaced apart forming second gaps between adjacent second petals; and
the first ring and the second ring being configured to interlock so that the first petals and second petals overlap.

13. The medical device of claim 12, wherein the first petals interleave with the second petals by filling the second gaps and the second petals interleave with the first petals by filling the first gaps.

14. The medical device of claim 12, wherein the first petals and the second petals are formed from a polymer material.

15. The medical device of claim 14, wherein the polymer material is taken from the group of polymers including polyamides, polyamide copolymers such as PEBAX, silicone rubber, polyurethanes, and nylons.

16. The medical device of claim 12, wherein at least a portion of the first petals and the second petals are formed from a metallic material.

17. The medical device of claim 12, wherein at least some of the first petals and second petals are loaded with a radiopaque material to enhance visualization of the device under fluoroscopy.

18. The medical device of claim 17, wherein the radiopaque material is barium sulfate.

19. The medical device of claim 12, wherein a radiopaque material is attached to at least some of the petals to enhance visualization of the device under fluoroscopy.

20. The medical device of claim 12, wherein the first petals and the second petals have a distal end and a proximal end, the first petals and the second petals tapering from a relatively narrower proximal end to a relatively wider distal end.

21. The medical device of claim 20, wherein the distal ends of the first petals and the second petals are biased radially outwardly to form a flared configuration.

22. The medical device of claim 12, wherein the first petals and the second petals are flexible so that the petals can be collapsed into a delivery configuration and flared radially outwardly into a deployed configuration.

23. The medical device of claim 12, wherein the deflector has a distal end having a first diameter in the delivery configuration and a second diameter in the deployed configuration.

24. The medical device of claim 23, wherein the deflector distal end first diameter is smaller than the second diameter.

25. An apparatus for delivering a cardiac harness onto a heart, comprising:
an elongate body having a proximal portion and a distal portion, the distal portion having a tubular housing sized to contain the cardiac harness in a compacted configuration, the tubular housing having a proximal end, an open distal end, an inner surface, and an outer surface;
a plurality of elongate push rods longitudinally movable with respect to the elongate body, the cardiac harness releasably connected to each of the push rods such that advancement of the push rods in a distal direction moves the cardiac harness from the compacted configuration in the housing to an expanded configuration outside the housing;
a deflector sheath slidably mounted over the outer surface of the tubular housing for retaining the petals; and
the petals being configured to be collapsed into the deflector sheath in a delivery configuration and flared radially outwardly in a deployed configuration so that the push rods and cardiac harness slide over the deflector as the harness slides over the heart.

26. The apparatus of claim 25, wherein the plurality of petals number in the range from four to twenty petals.

27. The apparatus of claim 25, wherein the petals are formed from a polymer material.

28. The apparatus of claim 25, wherein the polymer material is taken from the group of polymers including polyamides, polyamide copolymers such as PEBAX, silicone rubber, polyurethanes, and nylons.

29. The apparatus of claim 25, wherein the proximal ends of the petals are biased radially outwardly to form a flared configuration.

30. The apparatus of claim 25, wherein the housing has a substantially circular cross-sectional shape having a diameter, and wherein at least a portion of the housing is compressible to a substantially elliptical cross-sectional shape having a minor axis that is less than the diameter.

31. The apparatus of claim 25, wherein the housing has a cross-sectional shape having a first perimeter, and wherein at least a portion of the housing is compressible to a reduced cross-sectional shape having a second perimeter that is less than the first perimeter.

32. The apparatus of claim 25, wherein the cross-sectional shape is adapted for advancing through a minimally invasive surgical entry path.

33. The apparatus of claim 25, wherein the housing tapers from a first cross-sectional shape having a first perimeter at the proximal end of the housing to a second cross-sectional shape having a second perimeter at the distal end of the housing, the second perimeter being smaller in size than the first perimeter.

34. A method of delivering and mounting a cardiac harness on a heart, comprising:
providing a minimally invasive access site;
advancing an elongated delivery device through the access site;
extending a deflector out of the delivery device;
advancing a cardiac harness out of the delivery device and over the deflector;
the cardiac harness flaring outwardly as the harness advances over the deflector and onto the heart; and
withdrawing the delivery device and deflector through the access site.

35. The method of claim 34, wherein a suction cup extends from a distal end of the delivery device and engages the heart wherein a vacuum is drawn within the suction cup to firmly attach the cup onto the heart.

36. The method of claim 35, wherein the deflector extends over the suction cup.
